(19) United States
(12) Patent Application Publication
(10) Pub. No.: US 2024/0159659 A1
(43) Pub. Date: May 16, 2024

(54) DETERMINATION DEVICE, LEARNING DEVICE, DETERMINATION SYSTEM, DETERMINATION METHOD, LEARNING METHOD, AND PROGRAM

(71) Applicant: J-OIL MILLS, Inc., Tokyo (JP)

(72) Inventors: Kenya ITO, Tokyo (JP); Takeshi SUZUKI, Tokyo (JP); Masami INOUE, Tokyo (JP); Ryohei WATANABE, Tokyo (JP); Ayato TAKASAKI, Tokyo (JP); Kenichi KAKIMOTO, Tokyo (JP)

(21) Appl. No.: 18/281,738

(22) PCT Filed: Mar. 11, 2022
(86) PCT No.: PCT/JP2022/010963

§ 371 Date: Sep. 12, 2023

(30) Foreign Application Priority Data
Mar. 25, 2021 (JP) 2021-051744
May 18, 2021 (JP) 2021-084010

(51) Int. Cl. G01N 21/27; A47J 37/12; G01N 11/00; G01N 33/03; G06Q 50/12; G06T 7/00; G06V 10/70; G06V 20/68

(52) U.S. Cl. CPC G01N 21/27; A47J 37/1266; G01N 11/00; G01N 33/03; G06Q 50/12; G06T 7/0002; G06V 20/68; G06T 2207/20081; G06V 10/70

(57) ABSTRACT

Efficient acquisition of information to adjust a cooking environment for providing a fried food with good taste is realized by determining the cooking environment in advance based on a state of edible oil. A determination device for determining a cooking environment of an edible oil, comprising: an imaging section configured to acquire an image in which the edible oil is captured; a first input section configured to input first information which is information on a fried food to be fed into the edible oil and cooked; a first identification section configured to analyze the image and identify a state of the edible oil; and a second identification section configured to identify the cooking environment in which the fried food is to be cooked based on the first information and the state.

### TECHNICAL FIELD

The present invention relates to a determination device, a learning device, a determination system, a determination method, a learning method, and a program.

### BACKGROUND ART

Appropriately managing the quality of edible oil in cooking of deep-fried foods (hereinafter, referred to as “cooking”) preferably enables the quality of deep-fried foods to be kept.

Specifically, for example, a device for measuring the state of edible oil or oil and fat has been known. In deep-fry cooking of foods or the like, edible oil may be used over a long period of time. In addition, the temperature of the edible oil in use during the cooking reaches about 130° C. to 180° C. Then, oxidation due to oxygen or the like in the atmosphere deteriorates the edible oil. The deterioration of edible oil causes, for example, aldehydes, ketones, and polymer compounds to generate. These components adversely affect the taste and the like. In this respect, a sensor is used to measure the electrical properties of the edible oil. Techniques for measuring such properties using a sensor and protecting the sensor with coating have been known (see, for example, Patent Literature 1).

### CITATION LIST

### Patent Literature

Patent Literature 1: JP-A-2010-534841

### SUMMARY OF INVENTION

### Technical Problem

In the conventional techniques, in many cases, it is difficult to determine in advance how the environment where the cooking is to be performed (hereinafter, referred to as “cooking environment”) will be when a food to be deep-fried is put into the edible oil. The cooking environment which is not ready for the food to be deep-fried may impair the taste of the fried food thus cooked. Considering this, it is preferable to determine in advance the cooking environment after the food to be deep-fried is put into the edible oil, such as whether the cooking environment corresponds to an optimum cooking environment, or to what extent of use makes the frying oil become an unsuitable cooking environment. However, the conventional techniques have problems in efficient determination of a cooking environment and acquisition of information for adjusting the cooking environment.

An object of the present invention is to efficiently acquire information so as to adjust a cooking environment for providing a fried food with good taste by determining the cooking environment in advance based on a state of edible oil.

### Solution to Problem

In order to achieve the object described above, provided is a determination device for determining a cooking environment of an edible oil, comprising: an imaging section configured to acquire an image in which the edible oil is captured; a first input section configured to input first information which is information on a fried food to be fed into the edible oil and cooked; a first identification section configured to analyze the image and identify a state of the edible oil; and a second identification section configured to identify the cooking environment in which the fried food is to be cooked, based on the first information and the state.

### Advantageous Effects of Invention

According to the present invention, it is possible to efficiently acquire information so as to adjust a cooking environment for providing a fried food with good taste by determining the cooking environment in advance based on a state of edible oil.

### BRIEF DESCRIPTION OF DRAWINGS

FIG. 1 illustrates an example of arrangement in a cooking area 1.

FIG. 2 illustrates an example of a hardware configuration of an information processing device.

FIG. 3 illustrates an example of a scale 24.

FIG. 4 illustrates an example of entire processing.

FIG. 5 illustrates an example of entire processing of a configuration using AI.

FIG. 6 illustrates an example of entire processing of a configuration using a table.

FIG. 7 illustrates an example of identification of good taste.

FIG. 8 illustrates an example of a second embodiment.

FIG. 9 illustrates an example of an input and output relation according to the second embodiment.

FIG. 10 illustrates an example of an information system 200.

FIG. 11 illustrates an example of a network structure.

FIG. 12 illustrates an example of a function configuration.

FIG. 13 illustrates an example of thermographic data.

FIG. 14 illustrates an example of areas.

FIG. 15 illustrates an example where a fry basket 3 is present.

FIG. 16 illustrates a second example where a fry basket 3 is not present.

FIG. 17 illustrates a second example where a fry basket 3 is present.

FIG. 18 illustrates an example of processing of estimating the height of the surface of edible oil.

FIG. 19 illustrates an example of a boundary.

FIG. 20 illustrates an example of detection of a boundary.

### DESCRIPTION OF EMBODIMENTS

Hereinafter, an object to be cooked using edible oil is referred to as “fried food”. The fried foods are, for example, fried chickens, croquettes, French fries, tempuras, pork cutlets, or the like.

### First Embodiment

### (Example of Arrangement in Cooking Area 1)

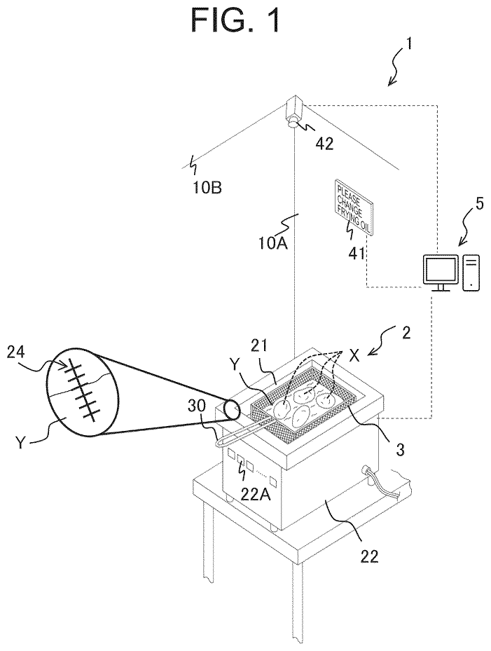

Firstly, an example of arrangement in a cooking area 1 where deep-fry cooking is performed to obtain the fried foods as listed above will be described with reference to FIG. 1.

FIG. 1 illustrates an example of arrangement in the cooking area 1. In the following, an example of edible oil is referred to as “frying oil”.

The cooking area 1 is built within a store such as a convenience store or a supermarket. The cooking area 1 is provided with cooking facilities for deep-fry cooking of fried foods X. The facilities include, for example, an electric fryer 2 (hereinafter, simply “fryer 2”).

The fryer 2 is a tool equipped with an oil vat 21, a housing 22, and the like.

The oil vat 21 stores frying oil Y therein. The oil vat 21 includes, for example, a handle 30, a fry basket 3, and the like.

The housing 22 accommodates the oil vat 21 therein. On a side surface of the housing 22, switches 22A serving as a setting operation unit for setting the temperature of the frying oil Y, the details of the deep-fry cooking, or the like are provided for each type of the fried foods X.

For deep-frying a food, firstly, a cook places a fried food X before deep-fried into the fry basket 3. Next, the cook hooks the handle 30 on an upper end portion of the housing 22 so that the fried food X before deep-fried is immersed in the frying oil Y. At the same time or around the same time, the cook presses one of the switches 22A in accordance with the type of the fried food X in cooking.

Upon passage of the time for completing the deep-fry cooking which corresponds to the one of the switches 22A as pressed, the fryer 2 notifies the cook of the completion of deep-frying. At the same time, the fryer 2 causes the fry basket 3 to rise from the oil vat 21 so that the fried food X immersed in the frying oil is pulled up therefrom.

For informing the completion of deep-fry cooking, for example, outputting a buzzer sound from a speaker, displaying the notification on a monitor 41 installed on a wall 10A, or the like may be employed. Thus, passage of the time for completing the deep-fry cooking is notified by means of light, sound, or a combination thereof.

The cook who is aware of the completion of deep-fry cooking of the fried food X pulls up the fry basket 3 to take the fried food X out therefrom. Note that the fry basket 3 may be automatically pulled up by a drive mechanism.

The arrangement in the cooking area 1 is not limited to the one with the tools as illustrated in FIG. 1. For example, the fryer 2 may be any type of tool capable of cooking, and may be arranged at a place other than the position illustrated in FIG. 1.

In the cooking area 1, an imaging device for capturing an image of the frying oil Y is installed. The imaging device is, for example, a video camera 42. Specifically, the video camera 42 is installed to a ceiling 10B or the like.

The video camera 42 captures the surface of the frying oil Y continuously to generate images thereof. The images are generated, preferably, in the form of a movie. The video camera 42 is installed with its condition, such as angle of view and focus, being adjusted.

Note that the video camera 42 does not necessarily have to be installed to the ceiling 10B. The video camera 42 may be installed to any position, such as the wall 10A, as long as the position allows the video camera 42 to capture an image of the frying oil Y.

Furthermore, the imaging device does not necessarily have to capture a movie. That is, for example, the imaging device may be a still camera, tablet, or the like for capturing still images. If using a still camera, the one capturing images intermittently along the time series may be adopted.

Furthermore, a plurality of imaging devices may be used. Still further, the imaging device may be a camera or the like equipped in a mobile device such as a tablet or a smartphone.

For example, a determination device 5 is configured with being connected to the monitor 41, the video camera 42, and the fryer 2. Note that the determination device 5 may not always be connected to the video camera 42, but may be configured to separately acquire an image captured by the video camera 42 and stored temporarily in a storage medium, and to execute the identification processing, etc.

The video camera 42 may be installed at a position other than the position illustrated in FIG. 1. Specifically, the video camera 42 can be installed, for example, at a position allowing it to capture an image of a scale 24.

### (Example of Hardware Configuration of Information Processing Device)

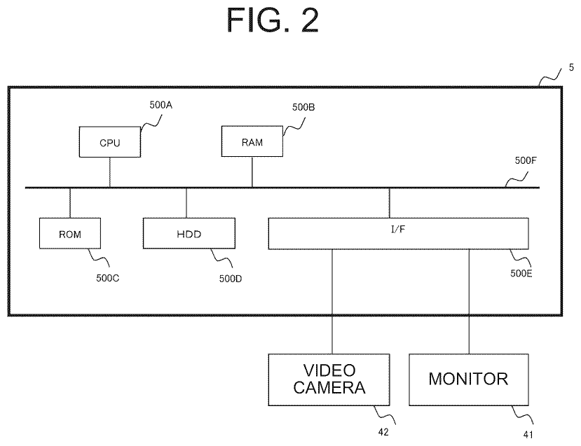

FIG. 2 illustrates an example of a hardware configuration of an information processing device. For example, the determination device 5 is an information processing device having the hardware resources as described below.

The determination device 5 includes a Central Processing Unit (hereinafter, referred to as “CPU 500A”), a Random Access Memory (hereinafter, referred to as “RAM 500B”), and the like. The determination device 5 further includes a Read Only Memory (hereinafter, referred to as “ROM 500C”), a hard disk drive (hereinafter, referred to as “HDD 500D”), interfaces (hereinafter, referred to as “I/F 500E”), and the like.

The CPU 500A is an example of a computing device and a control device.

The RAM 500B is an example of a main storage device.

The ROM 500C and the HDD 500D are examples of secondary storage devices.

The I/F 500E is provided for connection to an input device, an output device, or the like. Specifically, the I/F 500E connects an external device such as the monitor 41, the video camera 42, or the like by wire or wireless communication for inputting and outputting data.

Note that the hardware configuration of the determination device 5 is not limited to the one described above. For example, the determination device 5 may further include a computing device, a control device, a storage device, an input device, an output device, or a secondary device. Specifically, the information processing device may include a secondary device such as an internal or external Graphics Processing Unit (GPU).

Furthermore, a plurality of determination devices 5 may be provided.

### (Example of Identification of State)

For example, the determination device 5 identifies a state of edible oil (hereinafter, may be simply referred to as “state”) by means of the scale 24. Specifically, firstly, the video camera 42 captures an image such that the scale 24 is reflected therein. Then, the determination device 5 acquires the image in which the scale 24 appears from the video camera 42.

The scale 24 is provided on a wall surface of the oil vat 21. The scale 24 shows where the surface of the frying oil Y is located in the height direction. Accordingly, an image in which the scale 24 is captured together with the frying oil Y reveals the height of the surface of the frying oil Y. When the amount of oil per unit height is constant, the determination device 5 can calculates the amount of oil based on the height thus obtained. Accordingly, the scale 24 may be provided at any position so long as it can show the height or the like.

The scale 24 may be designed as described below.

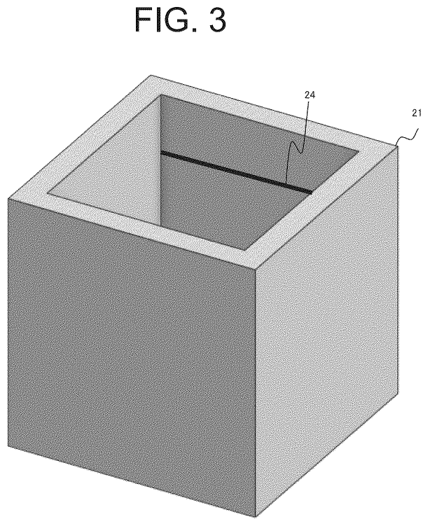

FIG. 3 illustrates an example of the scale 24. For example, as illustrated in FIG. 3, the scale 24 may be a line indicating an appropriate amount. Using the scale 24 as described above enables analysis, based on the image, how far the position of a net serving as the lower surface of the fryer 2 is from the scale 24.

More specifically, the scale 24 may be, for example, an “appropriate oil level line” described in “https://www.tanico.co.jp/category/maint/vol003/”.

The state may be other than the amount of oil. That is, a state other than the amount of oil may be analyzed based on an image. For example, the state may be the amount of oil, a difference from an optimum amount of edible oil (for example, the amount of oil capable of providing a fried food with the best taste, which is determined by experiments, etc.), the temperature of edible oil, or a combination thereof.

The state may be identified using other than an image. For example, a sensor other than the video camera 42 may be used to identify the state. Specifically, the sensor may include a flow meter, a weight meter, a stereo camera, a light field camera, and the like. Thus, the sensor is a device capable of ranging, measurement of the weight, or measurement of the amount of fluid.

Furthermore, the sensor may include a microphone, a thermometer, an odor sensor, etc.

The determination device 5 may acquire results of measurement by these sensors. Thus, combining a result of analysis of the image and results of measurement other than analysis of the image enables the determination device 5 to accurately identify the state such as the amount of oil.

### (Example of Entire Processing)

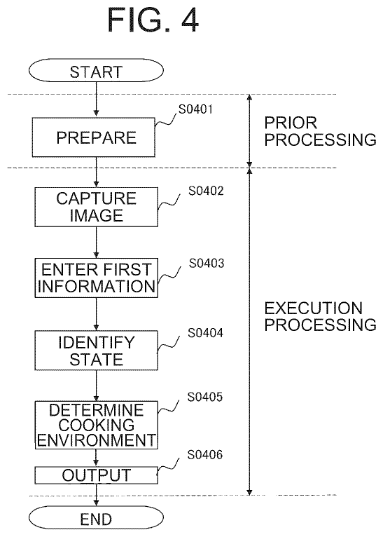

FIG. 4 illustrates an example of entire processing. For example, as illustrated in FIG. 4, the determination device 5 executes each processing in the order of “prior processing” and “execution processing”.

The prior processing is the processing executed in advance in order to prepare for the execution processing. Specifically, in the configuration using an artificial intelligence (hereinafter, referred to as “AI”) technology, the prior processing is the processing of causing the learning model to learn. The execution processing is the processing using a learned model prepared in the prior processing.

On the other hand, the execution processing may be the processing using a table or the like. In the configuration using a table, the prior processing is the processing of preparing such as entering the table (also referred to as a look-up table (LUT)), a formula, or the like. The execution processing is the processing using the table entered in the prior processing.

Note that the determination device 5 does not have to execute the prior processing and the execution processing in consecutive order as illustrated in FIG. 4. In other words, the period of time for preparation by the prior processing and the period of time for executing the execution processing thereafter do not necessarily have to be consecutive.

Accordingly, in the case of using AI, after a learned model was once created, the execution processing may be performed using the learned model in other opportunities.

Furthermore, when the learned model has been already generated, the determination device 5 may divert the learned model and omit the prior processing, and start the processing from the execution processing.

Still further, Transfer Learning, Fine tuning, or the like may be applied for a learning model and a learned model. An execution environment often varies for each device. Accordingly, while the basic configuration of AI is made learned in another information processing device, then further learning or setting may be performed by each determination device 5 for the purpose of optimization for each execution environment.

### (Example of Prior Processing)

In S0401, the determination device 5 performs preparation. The content of the prior processing differs depending on whether the configuration of the processing uses AI or a table.

In the configuration using AI, the determination device 5 performs preparation such as causing a learning model to learn or the like. On the other hand, in the configuration using a table, the determination device 5 performs preparation such as entering a table or the like. Details of the step of preparation will be described later.

### (Example of Execution Processing)

After execution of the prior processing, in other words, after completion of preparation of AI or the table, the determination device 5 performs the execution processing, for example, in the following procedures.

In step S0402, the determination device 5 acquires an image in which the edible oil is captured. As the image, a plurality of frames or images captured by a plurality of devices may be used. Hereinafter, such a plurality of images or a movie is simply referred to as “image”.

Preferably, the image is a color image. In other words, the image is preferably in a data-format such as RGB or YCrCb. Using a color image enables accurate analysis or recognition by colors.

In step S0403, the determination device 5 inputs first data.

The first information is the information about foods to be put into the edible oil for deep-fry cooking. Specifically, the first information is the information indicating the type of fried food, the amount of the fried foods to be put into the edible oil, or a combination thereof. Accordingly, the first information is input in such a manner that the name for distinguishing the type of fried foods, the number of fried foods to be put into the oil vat 21 in cooking, or the like is designated.

In step S0404, the determination device 5 analyzes the image to identify the state. In other words, the determination device 5 analyzes the image acquired in step S0402.

In step S0405, the determination device 5 determines a cooking environment.

In step S0406, the determination device 5 performs output based on the cooking environment or the like.

In the entire processing, the processing content up to step S0405 as described above differs depending on whether AI or a table is used in the configuration.

Hereinafter, each configuration will be described separately.

### (Entire Processing of Configuration Using AI)

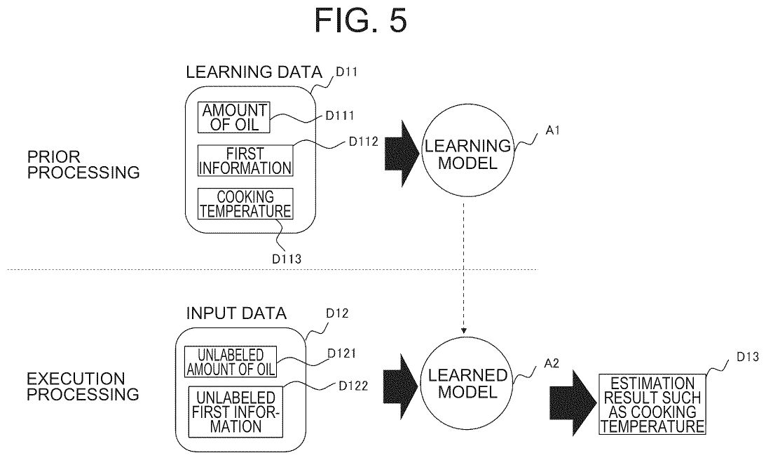

FIG. 5 illustrates an example of the entire processing of the configuration using AI. As illustrated in FIG. 5, in the configuration using AI, the prior processing is the processing of causing a learning model A1 to learn. The execution processing is the processing of determining a cooking environment or the like using a learned model A2, which is the learning model in which a certain degree of learning has been completed in the prior processing or the like.

The prior processing is, for example, the processing of causing the learning model to learn using learning data D11. In other words, the prior processing is the processing of causing the learning model A1 to learn by “supervised” learning using the learning data D11 to generate the learned model A2.

The learned data D11 is, for example, the data in which an amount of oil D111, first information D112, and a cooking temperature D113 are combined.

The amount of oil D111 is the amount of edible oil and the like. The amount of oil D111 is a result of analysis and the like obtained based on analysis of the image acquired in step S0402.

The amount of oil D111 is preferably obtained based on analysis of an image. In other words, the amount of oil D111 is preferably obtained by analyzing the image based on the scale 24 or the like and then input.

In many cases, information other than the amount of oil D111 can be obtained from an image. Accordingly, analyzing the image allows the determination device 5 to acquire the information other than the amount of oil D111. This may enable the determination device 5 to identify the state that affects the cooking environment and learn the state upon input of the image.

Furthermore, inputting the image allows the learning data to be obtained while the installed imaging device keeps imaging the edible oil continuously. This can simplify the preparation of the learning data more than the case of entering text data or the like.

The first information D112 is the information indicating the type of fried foods to be cooked under the condition of the state indicated by the amount of oil D111, the fed amounts of the fried foods, or a combination thereof. For example, the first data D112 may be input using text data or the like, or it may be obtained by analyzing an image and identifying the type of fried foods or the like based on image recognition, and then input.

The cooking temperature D113 is one of the examples of the cooking environment. In other words, the cooking temperature D113 is the information showing the cooking temperature D113 obtained as a result of cooking performed under the condition indicated by the first information D112. In other words, the cooking temperature D113 is the information showing how the cooking environment in which a fried food will be cooked become, and serves as “labeled training data” in the configuration for the “supervised” learning.

Note that the cooking environment is not limited to the cooking temperature D113. For example, the cooking environment may be a temperature allowing a fried food to be cooked, the amount of decrease in temperature when a fried food is fed into edible oil, the level of deterioration of edible oil, a combination thereof, or the like. As described above, the cooking environment is the information showing how the environment in which a fried food will be cooked become when using the current edible oil. Furthermore, the cooking environment may be determined, for example, based on a difference of how much the cooking environment deviates from the optimum cooking temperature. Alternatively, the cooking environment may be determined, for example, based on the degree in which the optimum cooking temperature cannot be maintained, in other words, to what extent of use of the current edible oil makes the frying oil become an unsuitable cooking environment.

Using the learning data D11 as described above and causing the learning model A1 to learn enables the determination device 5 to learn a relation between a combination of the state and the first information and the cooking environment. Then, using the learned model A2 generated by the learning above enables the determination device 5 to perform the execution processing as described below.

In the execution processing, input data D12 is input so that a result of estimation (hereinafter, simply referred to as “estimation result D13”) such as a cooking temperature is output.

For example, in the execution processing, the determination device 5 inputs the input data D12 including the state such as the amount of oil and the first information in the same manner as the prior processing. The execution processing differs from the prior processing in that the cooking environment that is to be the result with respect to the amount of oil and the first information has not been known yet.

Hereinafter, the amount of oil to be input by the executing processing is referred to as “unlabeled amount of oil D121”. In the same manner, the first information input by the executing processing is referred to as “unlabeled first information D122”.

The input data D12 is a combination of the unlabeled amount of oil D121 and the unlabeled first information D122, etc. The determination device 5 inputs the input data D12 to the learned model A2 (step S0403 and step S0404 in FIG. 4). In response to this input, the determination device 5 outputs the estimation result D13. In other words, how the cooking environment will become when the cooking is performed under the state indicated by the input data D12 is estimated (step S0405 in FIG. 4).

As described above, in the configuration using AI, even if the condition that differs from the condition input as the learning data D11 is input, the determination device 5 can estimate the cooking environment based on the learning.

### (Example of Entire Processing of Configuration Using Table)

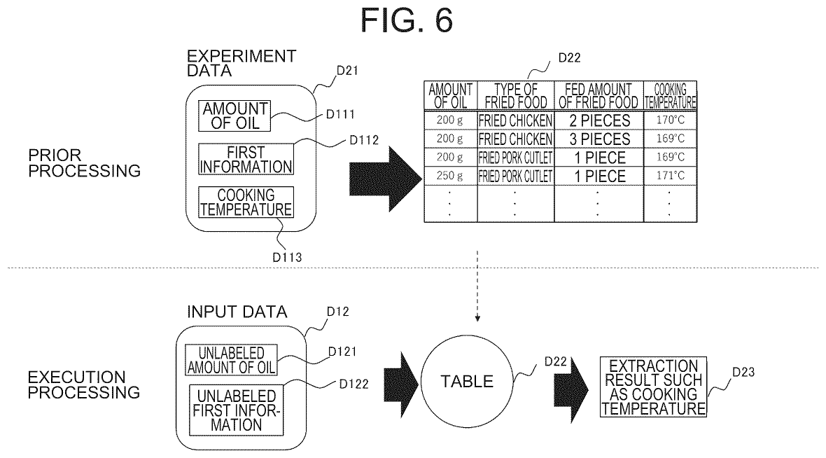

FIG. 6 illustrates an example of the entire processing of the configuration using a table. As illustrated in FIG. 6, in the configuration using a table, the prior processing is the processing of generating a table D22. The execution processing is the processing of determining the cooking environment or the like using the table D22 generated in the prior processing.

The prior processing is, for example, the processing of gathering experiment data D21 into the format of a table. Note that the table D22 may not be in the format of a two-dimensional table or the like as illustrated in FIG. 6. In other words, the table D22 can be in any data format or the like as long as it can uniquely identify the cooking temperature D113 corresponding to the amount of oil D111 and the first information D112.

The experiment data D21 is the data in which, for example, the data such as the amount of oil D111, the first data D112, and the cooking temperature D113 are combined. The amount of oil D111, the first information D112, and the cooking temperature D113 are, for example, the same as those in FIG. 5.

The table D22 is the data for associating the amount of oil D111, the first information D112, and the cooking temperature D113 with each other. The table D22 may include the information other than the information illustrated in FIG. 6.

Using the table D22 as described above enables the determination device 5 to associate the cooking environment with the combination of the state and the first information. Furthermore, using the table D22 as described above also enables the determination device 5 to perform the execution processing as follow.

For example, in the execution processing, the determination device 5 inputs the input data D12 including the state such as the amount of oil and the like and the first information in the same manner as the configuration using AI (step S0403 and step S0404 in FIG. 4).

In the same manner as the configuration using AI, in the configuration using a table as well, the execution processing differs from the prior processing in that the state such as the amount of oil and the first information are unknown.

In the following, in the same manner as the configuration using AI, an example in which the input data D12 is a combination of the unlabeled amount of oil D121 and the unlabeled first information D122 will be described.

In the same manner as the configuration using AI, the determination device 5 extracts the corresponding cooking temperature from the table D22 upon input of the combination of the state and the first information as the input data D12. Then, the determination device 5 outputs an extraction result D23 in response to the input. That is, in the same manner as the configuration using AI, the determination device 5 extracts, from the table D22, how the cooking environment will become when the cooking is performed under the condition indicated by the input data D12 (step S0405 in FIG. 4).

As described above, in the configuration using a table, the determination device 5 searches the cooking environment corresponding to the state that has been entered on the table D22, thereby realizing the high-speed processing.

The determination device 5 may estimate the cooking environment based on linear interpolation or the like. That is, under the condition that is not input in the table D22, the determination device 5 may calculate the cooking environment by averaging the similar conditions in the table D22 or the like.

For example, as illustrated in FIG. 6, when the “amount of oil is 200 g” and the “amount of oil is 250 g” have been entered in the table D22 and the “amount of oil is 225 g” (that is, has not been known) is a target of the execution processing, the determination device 5 may calculate an intermediate value between the “amount of oil is 200 g” and the “amount of oil is 250 g”.

Such interpolation enables the determination device 5 to address a condition that is not entered in the table D22.

### (Identification of Volume of Expansion and Example of Correction Based on Volume of Expansion)

Preferably, the determination device 5 identifies the volume of expansion and performs correction based on the volume of expansion. Hereinafter, the amount of edible oil before correction, in other words, the amount of edible oil obtained based on the result of analysis of an image is referred to as “first amount of oil”. On the other hand, the amount of edible oil after the first amount of oil is corrected based on the coefficient of expansion is referred to as “second amount of oil”.

The coefficient of expansion can be identified based on, for example, the type and temperature of edible oil. In other words, the determination device 5 can identify the coefficient of expansion of the edible oil that is a target of determination by identifying the type and temperature of the edible oil, etc.

The volume of edible oil changes with temperature. Furthermore, the coefficient of expansion differs depending the type of edible oil. Therefore, correcting the amount of oil while considering the coefficient of expansion enables the determination device 5 to accurately identify the amount of oil.

For example, the determination device 5 identifies the type of the edible oil based on analysis of the image, input of the name of the edible oil, or the like. Next, the determination device 5 measures and identifies the temperature of the edible oil or the like.

Furthermore, the determination device 5 inputs the data and the like in advance in which the set of the type of the edible oil and the temperature thereof is associated with the coefficient of expansion. Alternatively, the determination device 5 inputs a calculation formula or the like for calculating the coefficient of expansion in advance.

As described above, the determination device 5 identifies the first amount of oil based on analysis of the image or the like, and identifies the coefficient of expansion. Next, the determination device 5 corrects the first amount of oil to identify the second amount of oil. Specifically, the second amount of oil is calculated by the following formula (1).

Second amount of oil=First amount of oil×Difference in temperature×Coefficient of expansion (1)

In the formula (1) above, the “difference in temperature” is a value indicating how much the difference in temperature between the current temperature of the edible oil and the reference temperature is. Furthermore, in the formula (1) above, the “coefficient of expansion” is a value predetermined based on the set of the type of the edible oil and the temperature thereof. Thus, the determination device 5 performs the calculation by multiplying the first amount of oil by the difference in temperature and the coefficient of expansion to perform correction based on the coefficient of expansion.

The correction is not limited to use of the calculation by the formula (1) above. Any calculation method or the like may be employed as long as it can identify the amount of oil before expansion with eliminating the influence of expansion due to temperature. For example, the coefficient of expansion may be calculated using the specific gravity or the like.

Using the state including the second amount of oil determined based on the formula (1) above and the first information enables the determination device 5 to accurately identify the cooking environment and the like.

### (Example of Identification of Good Taste)

In step S0406 in FIG. 4, the determination device may further output a result of determination of good taste. In other words, the determination device identifies the good taste to be obtained in the cooking environment determined by step S0405.

The good taste is comprehensively evaluated in view of the oily taste, smell, texture, and flavor of a fried food. For example, the good taste is evaluated by sensory evaluation or the like.

The cooking environment is strongly correlated with good taste. Therefore, assuming the cooking environment enables the determination device to identify how a fried food will taste in the assumed cooking environment. In many cases, cooking a fried food at an optimum cooking temperature can improve its taste. Hereinafter, an example in which the cooking temperature is used as the cooking environment will be described.

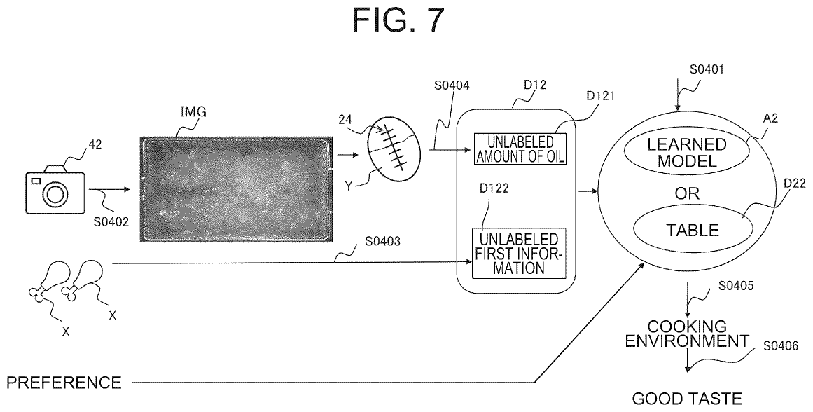

FIG. 7 illustrates an example of identification of the good taste. For example, an example in which the entire processing illustrated in FIG. 4 is performed will be described.

Upon completion of the prior preprocessing in step S0401, the determination device has been prepared for the learned model A2 or the table D22. After completion of the preparation, the determination device performs the execution processing.

In step S0402, the determination device acquires an image IMG.

In step S0403, the determination device inputs the unlabeled first information D122.

In step S0404, the determination device inputs the unlabeled amount of oil D121 based on the analysis of the image IMG.

Upon input of the input data D12 as described above, the determination device determines the cooking environment in step S0405.

The cooking environment is a cooking temperature, a temperature allowing the fried food X to be cooked, the amount of decrease in temperature when the fried food X is fed into edible oil, the level of deterioration of edible oil, a combination thereof, or the like. For example, determination of the cooking temperature enables the determination device to recognize the temperature at which the fried food X will be cooked. The cooking temperature is strongly correlated with the good taste for each type of fried food X, etc. Specifically, in many cases, a fried food which was not cooked at the optimum temperature does not taste good.

On the other hand, the cooking environment such as the cooking temperature can often be controlled to a temperature allowing the cooking to be performed depending on the amount of oil, an object to be cooked. The correlation between the amount of oil, the type of a fried food, and the like and the cooking environment is strong, and this enables the determination device to estimate the good taste based on the cooking temperature or the like.

Note that, as illustrated in FIG. 7, the determination device may consider preference. In other words, the determination device may estimate whether the taste of a fried food will match the input preference.

The preference of taste may differ from person to person. For example, in view of the oily taste, some people prefer the highly oil taste while others dislike. The preference shows an optimum value of an attribute included in the good taste described above.

In the case of inputting the preference, in the prior processing, it is preferable to input the labeled training data or enter, into the table, an attribute that is the target of preference so as to respond to the preference. Specifically, when inputting the oily taste as the preference in the execution processing, it is preferable to input the labeled training data or enter a result of the oily taste into the table.

However, each attribute has an item that is strongly correlated with the cooking environment depending on the type. That is, identifying the cooking environment may allow the determination device to determine whether a fried food can be cooked so as to respond to the preference. In this case, the determination device does not have to input an attribute that is the target of the preference as the labeled training data if grasping the relationship between the cooking environment and the attribute that is the target of the preference.

As described above, considering the preference enables the determination device to estimate the good taste depending on each person. Note that, in step S0406, the determination device may output, for example, how a fried food will taste under the input condition by means of a numerical value or a qualitative expression.

### Second Embodiment

The second embodiment is different from the first embodiment in that further oil to be added, waste oil, or the like is considered.

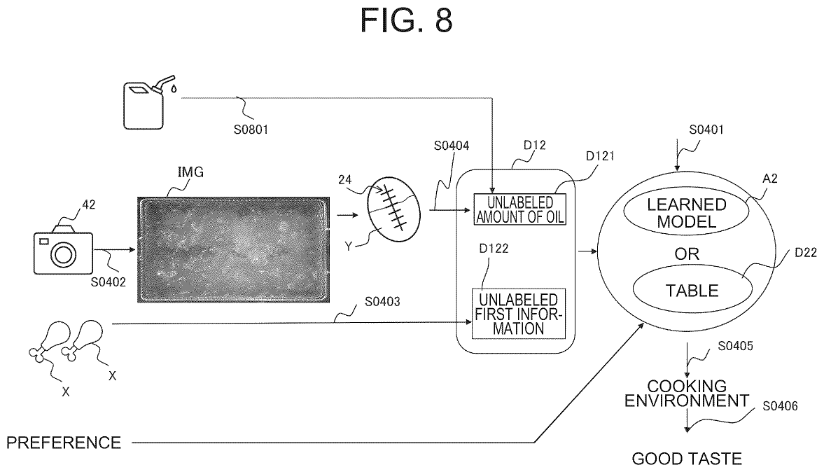

FIG. 8 illustrates an example of the second embodiment. In the second embodiment, the amount of oil is adjusted in step S0801, which is different from the example illustrated in FIG. 7.

In the second embodiment, for the amount of oil identified in step S0404, addition of edible oil, disposal of edible oil, or both is performed. This adjustment increases or decreases the amount of oil, and thus changes the unlabeled amount of oil D121. Considering this change, the determination device determines the cooking environment, the good taste, and the like. For example, in the second embodiment, input and output relationship is as follows.

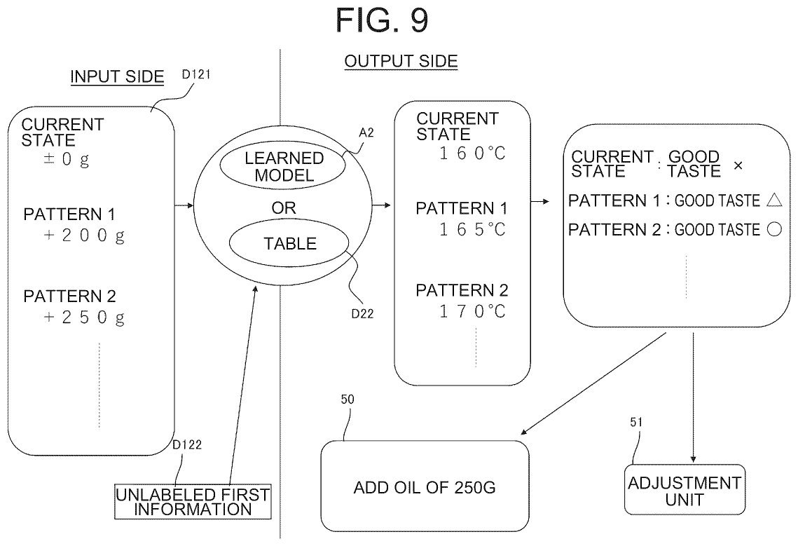

FIG. 9 illustrates an example of the input and output relationship according to the second embodiment. For example, a plurality of patterns of the unlabeled amount of oil D121 may be generated by adjustment.

Hereinafter, a pattern in which the amount of oil is not made increase or decrease, in other words, no adjustment is made and the current state is maintained is referred to as “current state”. On the other hand, “Pattern 1” and “Pattern 2” are examples in which adjustment is made, for example, by mixing further oil into the edible oil, in other words, adding edible oil.

Specifically, in “Pattern 1”, adjustment of adding the edible oil of “+200 g” to the “current state” is made. In “Pattern 2”, adjustment of adding the edible oil of “+250 g” to the “current state” is made.

Pattern other than the three patterns described above may be employed. In other words, patterns may be set such that parameters other than the amount of oil differ from each other. Specifically, the patterns may be set such that the first information differs among them. In the following, an example of making only the amount of oil differ for each pattern will be described.

Upon input of a plurality of patterns such as the “current state”, “Pattern 1”, and “Pattern 2”, the determination device determines the cooking environment for each of the patterns. Thus, the determination device can output the good taste for each cooking environment, in other word, for each pattern.

In the following, good taste is expressed using “o”, “Δ”, and “x” in descending order of evaluation. The good taste may be expressed using numerical values or words. Furthermore, the good taste may have attributes, and may be expressed using a result of evaluation for each of the attributes and a result of evaluation obtained by integrating the plurality of attributes.

Preferably, a pattern for making a fried food have the highest evaluation of the taste is output. For example, the determination device outputs a message 50 or the like informing an optimum pattern (in this example, “Pattern 2” showing the good taste of “o”). For example, the message 50 is output to a monitor or the like. Note that any format may be employed for the message 50.

Outputting the pattern for making the taste optimum can let the user know what kind of operation he or she should perform to provide the fried food with good taste.

Furthermore, the determination device may control an adjustment unit 51 or the like so as to realize an optimum pattern. For example, the adjustment unit 51 is a pump or the like. The determination device controls the adjustment unit 51 by outputting a signal for causing the pump to operate so that adjustment of the amount of oil can be performed in accordance with the operation of the pump.

As described above, the determination device may be configured to control the related equipment such as the adjustment unit 51 connected thereto so as to realize an optimum pattern. With this configuration, the determination device can adjust the cooking environment based on a result of determination of the cooking environment made in advance.

The determination device may generate further optimum patterns. That is, the determination device may extract an adjustment amount or the like for the current state so as to improve the taste more than the current state.

As described above, the determination device outputs the amount of oil to be added and amount of waste oil for achieving the optimal cooking environment or taste based on the cooking environment or a result of identification of the good taste.

Hereinafter, information indicating the amount of edible oil to be further added, amount of waste edible oil, or a combination thereof is referred to as “second information”.

Such output can let the user to know what kind of operations he or she should perform so as to provide a fried food with good taste.

Furthermore, the determination device may consider adjustment of an item other than the second information, in other words, adjustment of an item other than the amount of oil. For example, the determination device may also adjust the timing of adding further oil (hereinafter, referred to as “first timing”), the timing of disposing the oil (hereinafter, referred to as “second timing”), and the like.

In cooking, adjustment of the cooking environment may be affected by the timing of adding further edible oil or disposing the oil. Accordingly, the determination device may estimate the first timing and the second timing for realizing the cooking environment in which a fried food with better taste can be provided.

Such output of the first timing, the second timing, and the like which are optimized can let the user to know what kind of operations he or she should perform so as to provide a fried food with good taste.

Alternatively, the determination device may control the adjustment unit 51 or the like to adjust the edible oil at the optimum first timing and the optimum second timing.

Furthermore, as described below, the determination device may provide information about adjustment or the like in the information system.

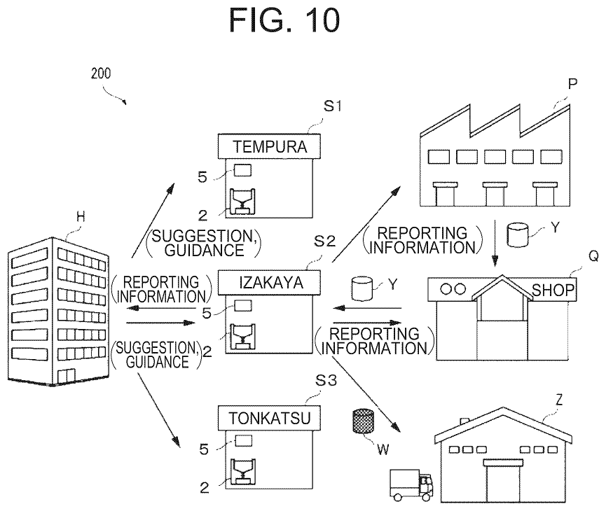

FIG. 10 illustrates an example of an information system 200. For example, the determination devices 5 installed in shops S1 to S3, respectively, are connected using a communication line or the like so as to configured the information system 200.

For example, the shop S2 (in this example, izakaya) notifies a headquarters H with reporting information. In this case, the headquarters H analyzes the number of times or frequency of receiving the reporting information. The headquarters H analyses in the same manner for the shop S1 (in this example, tempura restaurant) or the shop S3 (in this example, tonkatsu restaurant).

Based on the result of analysis thus obtained, the headquarters H provides suggestions or guidance as to whether the edible oil is appropriately used, appropriately changed, and efficiently used.

The headquarters H may manage the factories in which the fryers 2 are installed. The headquarters H may also manage each fryer 2 installed in equipment of the stores, shops, or factories.

A manufacturer P of edible oil and a seller Q of edible oil are also notified of the reporting information. Upon receiving the reporting information, the manufacturer P forms a manufacturing plan or a sales plan for edible oil. Upon receiving the reporting information, the seller Q orders and purchases edible oil from the manufacturer P. Then, the seller Q distributes the edible oil to the shop S1, shop S2, and shop S3.

Still further, a disposal company Z (note that the disposal company Z and the manufacturer P may be the same) of edible oil is also notified of the reporting information. Upon receiving the reporting information, the disposal company Z arranges collection of waste oil W. Specifically, when receiving the reporting information for a predetermined number of times, an operator from the disposal company Z visits the shop S2 to collect the waste oil W from the oil vat 21 of the fryer 2.

Still further, a cleaning company (not illustrated) may also be notified of the reporting information. Upon receiving the reporting information, a cleaning operator visits the shop S2 to clean the inside of the oil vat 21 of the fryer 2 and therearound.

Thus, using the reporting information enables quick operations including supply of edible oil, disposal thereof, and cleaning in the shops S1 to S3.

Furthermore, automating the change of edible oil in the shops and stores enables reduction in the burden on a user (employee in the shops and stores). Specifically, outputting the reporting information indicating that the rate of deterioration of the edible oil exceeds a threshold value causes the edible oil in use to be changed to new one.

In the supply chain as described above, when the oil is to be adjusted, for example, by addition of oil or disposal of oil, the determination device 5 may notify the headquarters H, the disposal company Z, the manufacturer P of the amount of edible oil and the time when the oil is to be added or disposed. Thus, for addition of oil or disposal of oil, automating ordering, collection, delivery, and procedures by the information system 200 enables the user to reduce his or her workload.

### (Example of Network Configuration)

AI is implemented by, for example, the following network.

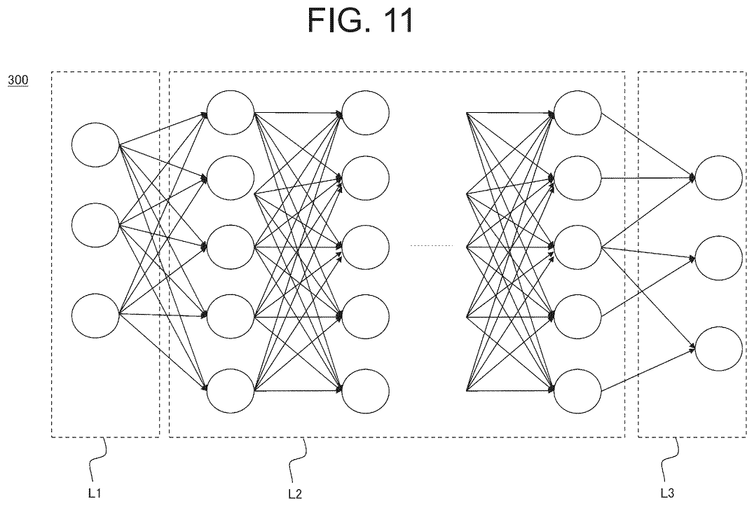

FIG. 11 illustrates an example of a network structure. For example, each of the learning model and the learned model has a network 300 having the structure as described below.

The network 300 includes, for example, an input layer L1, an intermediate layer L2 (also referred to as “hidden layer”), and an output layer L3.

The input layer L1 is a layer for inputting data.

The intermediate layer L2 converts the data input in the input layer L1 based on weights, biases, and the like. Thus, a result obtained by the process in the intermediate layer L2 is transmitted to the output layer L3.

The output layer L3 is a layer for outputting a result of estimation, etc.

Coefficients of the weights and the like are optimized by learning. Note that the network 300 is not limited to the network structure illustrated in FIG. 37. In other words, AI may be implemented by other machine-learning.

### (Level of Deterioration)

The level of deterioration is, for example, an acid value of edible oil, viscosity of edible oil, rate of increase in viscosity of edible oil, color tone of edible oil, Anisidine value of edible oil, quantity of polar compounds of edible oil, Carbonyl value of edible oil, smoke point of edible oil, Tocopherol content of edible oil, iodine value of edible oil, refractive index of edible oil, quantity of volatile compounds of edible oil, composition of volatile compounds of edible oil, flavor of edible oil, quantity of volatile compounds of a fried food obtained by deep-fry cooking with edible oil, composition of volatile compounds of a fried food, flavor of a fried food, or combination thereof.

An acid value (may be referred to as “AV”) of edible oil is a value measured by a method according to, for example, the standard methods for the analysis of fats, oils and related materials, 2.3.1-2013.

A rate of increase in viscosity of edible oil is, for example, a value calculated using the ratio of the amount of increase in viscosity relative to a reference value that is, for example, the viscosity of new edible oil before being used in deep-fry cooking for the first time after changing of oil (that is, viscosity at the start of use). Note that the viscosity is measured by a viscometer or the like. The viscometer is, for example, an E-type viscometer (TVE-25H, made by Toki Sangyo Co., Ltd.).

The color tone of edible oil (may be referred to as “color” or “hue”) is a value measured by a method according to, for example, the standard methods for the analysis of fats, oils and related materials, 2.2.1.1-2013. (for example, using a yellow component value and a red component value, a value is calculated by “the yellow component value plus 10×the red component value”).

An Anisidine value of edible oil is a value measured by a method according to the standard methods for the analysis of fats, oils and related materials, 2.5.3-2013.

The quantity of polar compounds of edible oil is a value measured by a method according to the standard methods for the analysis of fats, oils and related materials, 2.5.5-2013. For example, the quantity of polar compounds of edible oil is a value measured by a polar compound measurement device (such as the one made by Testo K.K.).

A Carbonyl value of edible oil is a value measured by a method according to, for example, the standard methods for the analysis of fats, oils and related materials, 2.5.4.2-2013.

A smoke point of edible oil is a value measured by a method according to the standard methods for the analysis of fats, oils and related materials, 2.2.11.1-2013. Smoke generates due to combustion of lipids contained in edible oil or decomposed products thereof.

The Tocopherol content of edible oil (may referred to as “vitamin E”) is the content of Tocopherol contained in the edible oil. Tocopherol is a value measured by a method according to, for example, a High Performance Liquid Chromatography (HPLC) method.

An iodine value of edible oil indicates, for example, the grams of iodine that can be added to 100 grams of oil or fat. An iodine value of edible oil is a value measured by a method according to, for example, the standard methods for the analysis of fats, oils and related materials, 2.3.41-2013.

A refractive index of edible oil is a value measured by a method according to, for example, the standard methods for the analysis of fats, oils and related materials, 2.2.3-2013.

The quantity of volatile compounds of edible oil, the composition of volatile compounds of edible oil, the quantity of volatile compounds of a deep-fried food obtained by deep-fry cooking with edible oil, and the composition of volatile compounds of a deep-fried food are defined by components (mainly odor components) volatilized from the deep-fried food or the edible oil. In the edible oil, the quantity or composition of the volatile components changes with deterioration of the edible oil. The volatile components may be measured by, for example, a Gas Chromatograph-Mass Spectrometer (GC-MS) or an odor sensor.

The flavor of edible oil and the flavor of a deep-fried food are values measured by sensory evaluation (for example, evaluation by a person who has actually eaten the deep-fried food) or a taste sensor.

### (Example of Function Configuration)

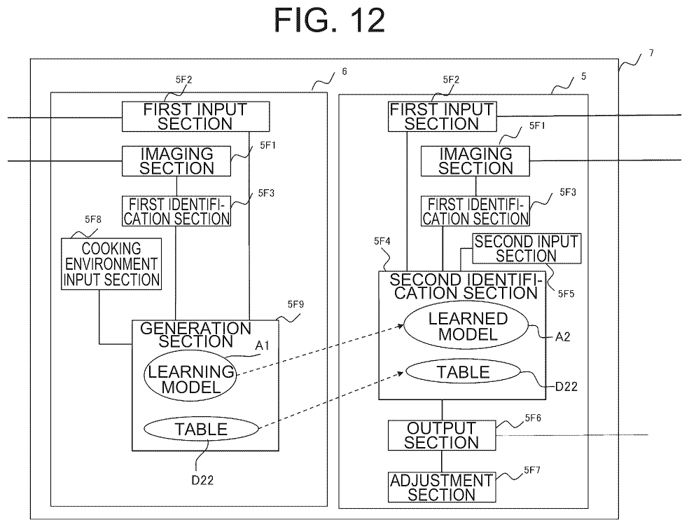

FIG. 12 illustrates an example of a function configuration. For example, the determination device 5 comprises a function configuration including an imaging section 5F1, a first input section 5F2, a first identification section 5F3, a second identification section 5F4, etc. Note that, as illustrated in FIG. 12, preferably, the determination device 5 comprises a function configuration further including a second input section 5F5, an output section 5F6, an adjustment section 5F7, etc. Hereinafter, an example of the function configuration illustrated in FIG. 12 will be described.

The imaging section 5F1 performs an imaging process of acquiring an image in which the edible oil is captured. For example, the imaging section 5F1 is implemented by the video camera 42, the I/F 500E, etc.

The first input section 5F2 performs a first input process of inputting the first data. For example, the first input section 5F2 is implemented by the I/F 500E, etc.

The first identification section 5F3 analyzes the image and performs a first identification process of identifying a state. For example, the first identification section 5F3 is implemented by the CPU 500A, etc.

The second identification section 5F4 performs a second identification process of identifying a cooking environment in which a fried food is to be cooked, based on the first information and the state. For example, the second identification section 5F4 is implemented by the CPU 500A, etc.

The second input section 5F5 performs a second input process of inputting the second information indicating the amount of oil to be added, amount of waste oil, or a combination thereof. For example, the second input section 5F5 is implemented by the I/F 500E, etc.

The output section 5F6 performs an output process of outputting the cooking environment, the amount of oil to be added for optimizing the taste, the amount of waste oil, the first timing, the second timing, or a combination thereof, based on the cooking environment and a result of identification of the good taste. For example, the output section 5F6 is implemented by the I/F 500E, etc.

The adjustment section 5F7 performs an adjustment process of addition or disposal of edible oil or both based on a result of output from the output section 5F6. For example, the adjustment section 5F7 is implemented by the adjustment unit 51, etc.

For example, the determination system 7 including the determination device 5 and the learning device 6 has the following function configuration. Hereinafter, an example where the learning device 6 has the same hardware configuration as that of the determination device 5 will be described. However, the determination device 5 and the learning device 6 may have different hardware configurations from each other.

In the same manner as the determination device 5, the learning device 6 comprises, for example, a function configuration including the imaging section 5F1, the first input section 5F2, the first identification section 5F3, etc. However, the learning device 6 can employ any configuration for input or data format as long as it can input the state and the first information. Hereinafter, the same function configurations as those of the determination device 5 will be provided with the same reference signs, and explanation therefor will be omitted.

A cooking environment input section 5F8 performs a cooking environment input process of inputting a cooking environment in which a fried food is to be cooked. For example, the cooking environment input section 5F8 is implemented by the I/F 500E, etc.

The generation section 5F9 performs a generation process of generating the learned model A2 by causing the learning model A1 to learn. Alternatively, the generation section 5F9 performs a generation process of generating the table D22. For example, the generation section 5F9 is implemented by the CPU 500A, etc.

In the determination system 7, the learned model A2 or the table D22 generated by the learning device 6 is distributed from the learning device 6 to the determination device 5 or the like via a network, etc.

The learning device 6 generates the learned model A2 or the table D22 by the prior processing. Generating the learned model A2 or table D22 enables the determination device 5 to determine the cooking environment in advance based on the state of the edible oil by the execution processing. When such a result of determination, in other words, information such as how the cooking environment will become, or which part is deviated from the optimum cooking environment is available before cooking, the cooking environment for providing a fried food with good taste can be easily adjusted.

Furthermore, compared with a case of adjusting the amount of oil or the like through trial and error or the like, the user is allowed to efficiently acquire the information such as the optimum approach to adjust the cooking environment.

Adjusting a cooking environment, for example, by reducing a deviation from the optimum cooking environment based on a result of determination as described above enables a delicious fried food to be provided.

### Third Embodiment

The determination device 5 may generate thermographic (thermography) data for use.

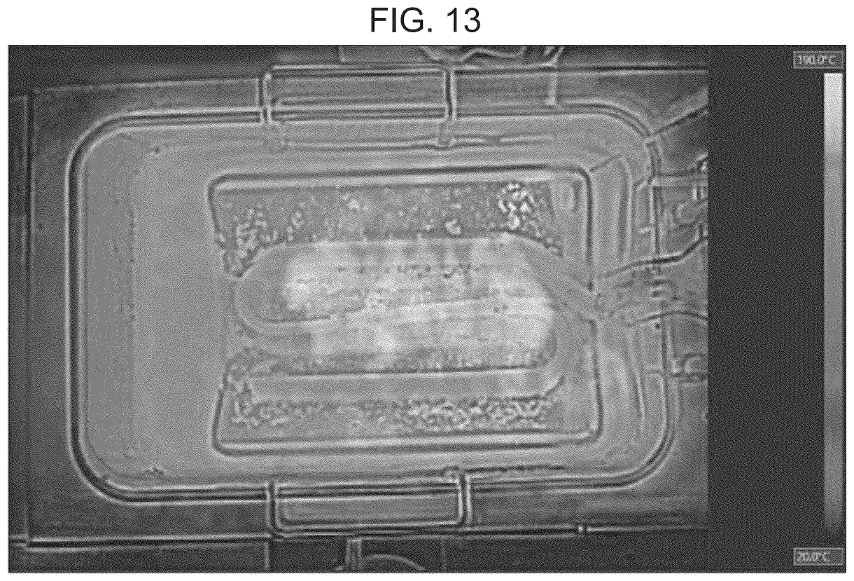

FIG. 13 illustrates an example of thermographic data. The thermographic data illustrated in FIG. 13 is an example of data generated by measuring the temperature of edible oil in a setting in which the edible oil is to be heated to 180° C. and “FLIR E4 made by FLIR (registered trademark) Systems” as a measurement device.

The thermographic data is the data indicating a temperature distribution of the edible oil in color. For example, thermographic data is generated by measuring infrared rays emitted from edible oil and plotting the temperature for each measurement point with color pixels. The example illustrated in FIG. 13 is an example of thermographic data showing the temperature in the range of 20° C. to 190° C. in color coding.

Thus, when the distribution of the temperature of edible oil during being heated is available, the expansion coefficient can be accurately identified. The expansion coefficient varies depending on the temperature. On the other hand, the temperature is not always uniformly distributed in the edible oil. In other words, the temperature of edible oil may vary depending on positions therein. However, using the thermographic data enables the determination device 5 to obtain the temperature for each position even when the temperature of the edible oil is not uniform as described above, thereby realizing identification of the expansion coefficient with accuracy.

Note that, for example, the temperature may be measured for each region (hereinafter, referred to as “area”) set in advance as described below.

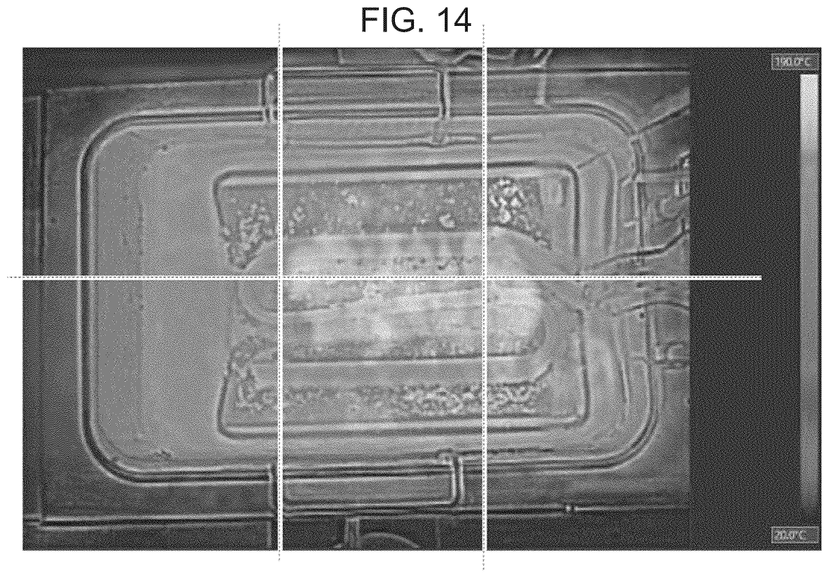

FIG. 14 illustrates an example of areas. FIG. 14 illustrates an example of a setting in which the entire regions of FIG. 13, where the temperature is to be measured, is divided into six areas. Note that an example of dividing the regions is not limited to the example illustrated in FIG. 14. That is, the entire regions may not be equally divided, or may be divided into areas other than six.

Not the entire regions do not have to be divided. For example, preferably, within the entire regions, an area including edible oil but not including any heating wire is extracted and set. In other words, it is preferable to set an area while avoiding a portion including a heating wire. A portion including a heating wire may be measured high depending on the temperature of the heating wire. Accordingly, setting an area so as to exclude a high-temperature portion such as a portion including a heating wire allows the temperature to be measured accurately.

When the scale 24 can be checked using an image, preferably, an area including the scale 24 is extracted and set. In other words, an area is preferably set for a region in which the scale 24 can be seen.

Furthermore, preferably, a region including only the edible oil is extracted and the temperature is measured for the extracted region. In other words, when a range in which the temperature is to be measured includes an object other than the edible oil, preferably, a result excluding a result of measurement obtained by measuring the object other than the edible oil is used.

In this example, the temperature in the six areas is measured separately. Accordingly, the determination device 5 identifies an expansion coefficient and the like for each area. For example, the determination device 5 performs statistical processing on a plurality of results of measurement indicated by the thermographic data for each area (for example, averaging the results of measurement belonging to the areas, and the like) to identify the temperature for each area.

When the temperature is measured for each area as described above, thermometers may be installed in the areas, respectively, to measure the temperature.

As described above, preferably, the determination device 5 further includes a temperature measurement section. This allows the determination device 5 to identify an expansion coefficient for each result of measurement indicating a distribution of the temperature of the edible oil.

As described above, considering a distribution of the temperature enables the determination device 5 to accurately identify an expansion coefficient.

### Fourth Embodiment

Preferably, the determination device 5 considers the fry basket 3 and the like as described below. For example, the thermographic data illustrated in FIG. 13 is the one when the fry basket 3 is not present. On the other hand, in the following, the thermographic data when the fry basket 3 is present will be described.

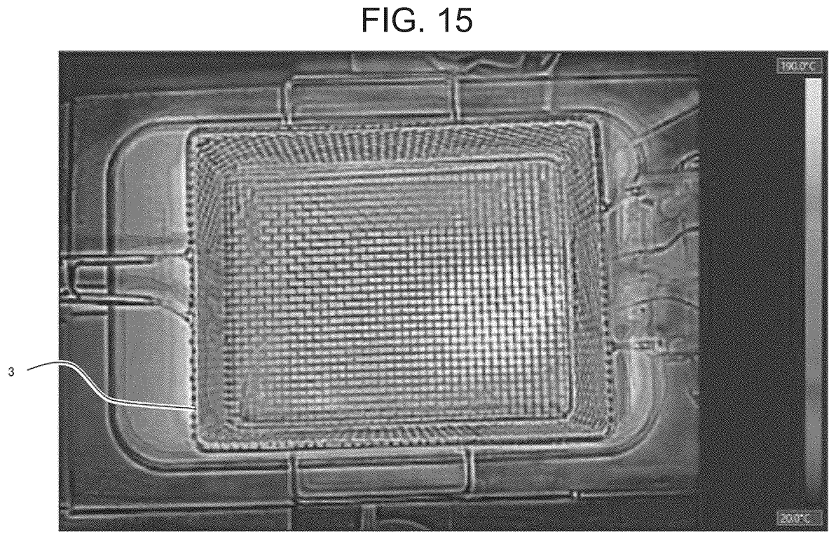

FIG. 15 illustrates an example where the fry basket 3 is present. FIG. 15 differs from FIG. 13 in that the fry basket 3 is present. On the other hand, both the setting in FIG. 13 and that in FIG. 15 are the one in which edible oil is to be heated to 177° C.

As illustrated in FIG. 15, when the fry basket 3 is present, a result of measurement of the temperature tends to be low even under the same heating condition as the case without the fry basket 3 as illustrated in FIG. 13. Specifically, when the fry basket 3 is present, due to the temperature of the fry basket 3, low-temperature portions increase within a temperature distribution. As a result, even if trying to identify the temperature based on the position of the edible oil using the thermographic data in the same manner as the example illustrated in FIG. 13, the temperature is determined at a low value. This tendency is the same even if the condition of the fryer or the like is changed.

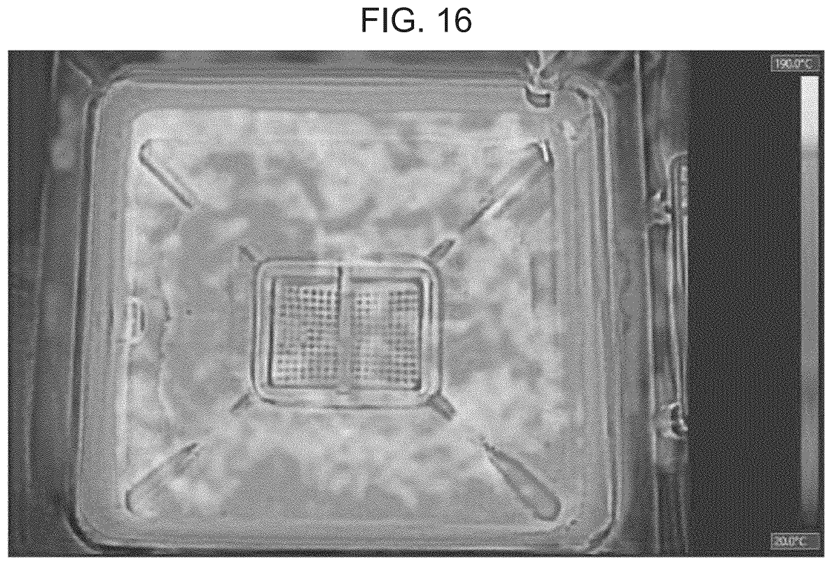

FIG. 16 illustrates a second example in which the fry basket 3 is not present.

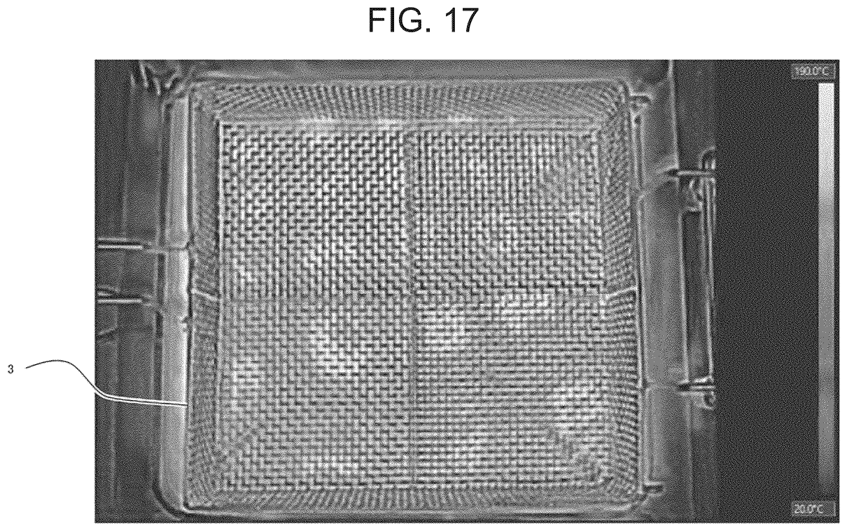

FIG. 17 illustrates a second example in which the fry basket 3 is present.

In FIG. 13 and FIG. 15, the fryer has a capacity of 3 liters while, in FIG. 16 and FIG. 17, the fryer has a capacity of 7 liters. In addition, FIG. 13 and FIG. 15 employ a heating condition of 177° C. while FIG. 16 and FIG. 17 employ a heating condition of 180° C.

On the other hand, FIG. 16 and FIG. 17 are different from each other in terms of whether the fry basket is present.

In the cases of FIG. 16 and FIG. 17, a temperature difference can be also found depending on whether the fry basket 3 is present.

For example, the determination device 5 recognizes whether the fry basket 3 is present by the processing such as recognizing the shape or the like based on an image or thermographic data. Note that whether the fry basket 3 is present may be recognized based on the weight, an operation by a user, or the like.

When determining that the fry basket 3 is present, the determination device 5 may correct a result of measurement of temperature. In other words, in identifying an expansion coefficient or the like, the determination device 5 may recognize the temperature to be lowered due to presence of the fry basket 3 in advance, and, when determining that the fry basket 3 is present, correct the temperature corresponding to the decreased amount.

However, even if it is determined that the fry basket 3 is present, for example, when the surface of the edible oil can be seen, the temperature may be measured accurately.

For example, when the surface of the edible oil cannot be seen, or when a deviation from a result of measurement obtained by the measurement device for measuring the temperature on the side of the fryer is found, the determination device 5 may correct the result measurement of temperature based on the thermographic data.

Furthermore, the determination device 5 may estimate the height of the surface of edible oil using the temperature difference caused by presence of the fry basket 3.

For this purpose, the determination device 5 is equipped with a measurement device for measuring the temperature on the side of the fryer, separately from the measurement device for generating thermographic data. Hereinafter, a measurement device for measuring the temperature on the side of the fryer will be referred to as “first measurement device” while a measurement device for measuring the temperature for thermographic data will be referred to as “second measurement device”.

There may be a difference in temperature between a result of measurement by the first measurement device (hereinafter, referred to as “first measurement result”) and a result of measurement by the second measurement device (hereinafter, referred to as “second measurement result”) due to the fry basket 3 or the like.

In addition, when the fry basket 3 is present, the fry basket 3 may make the scale 24 illustrated in FIG. 3 difficult to be seen from the video camera 42 as illustrated in FIG. 1.

In such cases, the determination device 5 estimates the height of the surface of edible oil based on the difference in temperature between the first measurement result and the second measurement result.

Thermographic data often tends to show the measured temperature low when the amount of edible oil is small. Therefore, when the amount of edible oil is small, a difference in temperature between the first measurement result and the second measurement result tends to be large. This enables, using a difference in temperature between the first measurement result and the second measurement result, estimation of the height of the surface of edible oil.

As described above, the determination device 5 further includes a first temperature measurement section for measuring the temperature of edible oil in the oil vat in which the edible oil is stored, and a second temperature measurement section for measuring the temperature of the edible oil from the surface of the edible oil. Then, the determination device 5 identifies a difference in temperature between the first measurement result that is a result of measurement by the first temperature measurement section and the second measurement result that is a result of measurement by the second measurement result.

The determination device 5 further includes a determination section configured to determine whether a cooking tool such as the fry basket 3 is present.

Next, when determining that a cooking tool is present, the determination device 5 estimates the height of the surface of edible oil based on a difference in temperature between the first measurement result and the second measurement result. Estimating the height of the surface of edible oil as described above enables the determination device 5 to accurately estimate the height of the surface of the edible oil.

Note that an experiment for obtaining the information on how much the height of the surface of edible oil varies depending on a difference in temperature of 1° C. is conducted in advance, and the information as obtained is input in the determination device 5.

### Fifth Embodiment

Thermographic data may be used to estimate the height of the surface of edible oil.

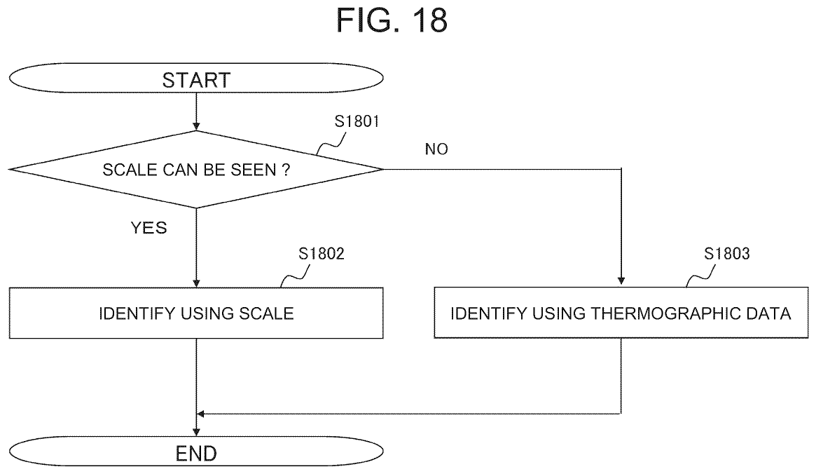

FIG. 18 illustrates an example of processing of estimating the height of the surface of edible oil. For example, the processing as illustrated in FIG. 18 is performed before the processing of identifying the height of the surface of edible oil, in other words, before identifying the amount of oil.

In step S1801, the determination device 5 determines whether the scale can be seen.

Whether the scale can be seen is determined based on, for example, whether the fry basket 3 is present. Specifically, for example, as illustrated in FIG. 17, when it can be determined that the fry basket 3 is present, the determination device 5 determines that the scale cannot be seen (NO in step S1801).

On the other hand, as illustrated in FIG. 16, when it can be determined that the fry basket 3 is not present, the determination device 5 determines that the scale can be seen (YES in step S1801).

Note that the determination device 5 may be configured to determine whether the scale can be seen based on a condition other than whether the fry basket 3 is present. For example, the scale 24 may be difficult to be seen due to a malfunction of the camera 42, polymers, bits of fried batter, or the like. Accordingly, the determination device 5 may determine that the scale cannot be seen when the scale 24 is not recognized as a result of image recognition on the scale 24 performed for an image (NO in step S1801).

The determination of whether the scale can be seen may be made based on a result of estimation. Specifically, the determination device 5 may estimate whether a line at which the temperature abruptly changes is present so as to determine whether the scale can be seen, as described below.

For example, when it can be determined that a line at which the temperature abruptly changes is present as described below, the determination device 5 may determine that the scale can be seen (YES in step S1801). In other words, when it can be determined that a line at which the temperature abruptly changes is present, the determination device 5 may directly identify the position of the oil level.

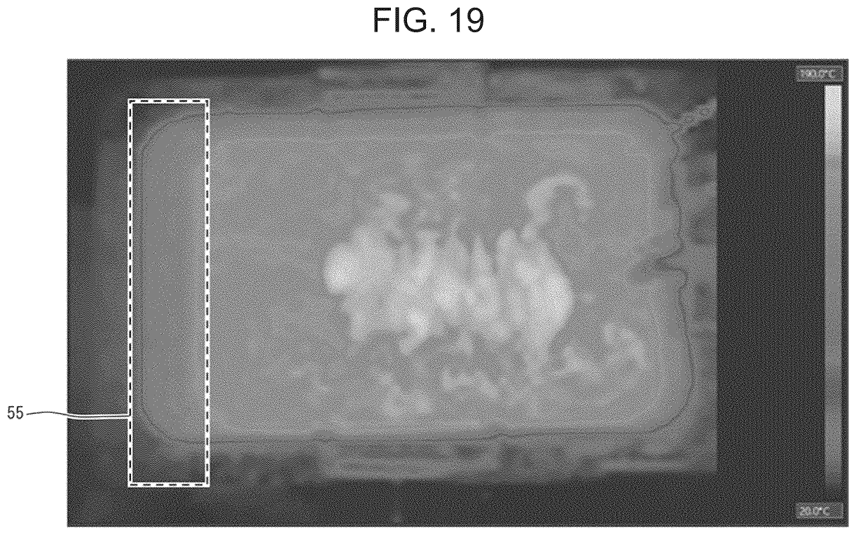

FIG. 19 illustrates an example of a boundary. For example, a boundary 55 is a line at which the temperature abruptly changes.

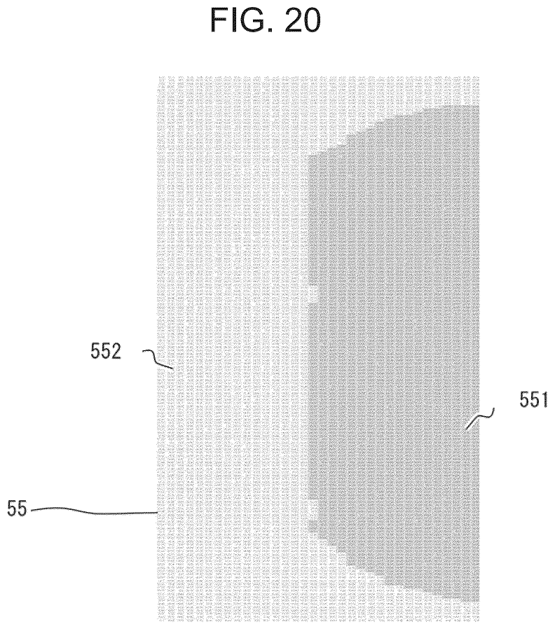

FIG. 20 illustrates an example of detection of a boundary. In FIG. 20, an example of a result of measurement of the temperature near the boundary 55 for each pixel. In this example, the determination device 5 determines that a line that separates pixels in which a relatively high temperature is measured (hereinafter, referred to as “high-temperature pixels 551”) and pixels in which a low temperature is measured (hereinafter, referred to as “low-temperature pixels 552”) as compared with the high-temperature pixel 551 is present.

In this example, a temperature measured in the high-temperature pixels 551 is equal to or higher than “160° C.”. On the other hand, a temperature measured in the low-temperature pixels 552 is less than “160° C.”.

The determination device 5 determines that the temperature abruptly changes to a threshold value or more at portions where the high-temperature pixels 551 and the low-temperature pixels 552 are adjacent to each other. The determination device 5 recognizes such portions as the boundary 55. Note that the threshold value for determining the boundary 55 is a value set in advance.

When determining that the boundary 55 is present as described above, the determination device 5 determines that the scale can be seen (YES in step S1801).

Next, when determining that the scale can be seen (YES in step S1801), the determination device 5 proceeds to step S1802. On the other hand, when determining that the scale cannot be seen (NO in step S1801), the determination device 5 proceeds to step S1803.

In step S1802, the determination device 5 identifies the height of the surface of edible oil using the scale.

In step S1803, the determination device 5 uses the thermographic data to determine the height of the surface of edible oil. In other words, the determination device 5 estimates the height of the surface of edible oil based on a difference in temperature between the first measurement result and the second measurement result.

Thus, switching the processing for identifying the height of the surface of edible oil based on whether the scale can be seen enables the determination device 5 to accurately identify the height of the surface of edible oil.

As described above, the determination device 5 identifies the height of the surface of edible oil using the scale when it can be determined that the scale can be seen, for example, when the scale can be recognized using an image or when a line at which the temperature abruptly changes is present (step S1802). For identifying the height of the surface of edible oil using the scale, the determination device 5 may use thermographic data.

On the other hand, when it can be determined that the scale is difficult to be seen, the determination device 5 identifies the height of the surface of edible oil using thermographic data (step S1803).

However, the amount of edible oil may be identified by a plurality of types of processing. For example, the determination device 5 may identify the height of the surface of edible oil using thermographic data even when the scale can be seen or the like. When performing the plurality of types of processing, the determination device 5 may perform statistical processing such as averaging on a plurality of results of processing to finally identify the amount of edible oil.

### (Modifications)

A part or all of the parameters may be acquired through data other than an image, an input operation by a user, or the like.

In the determination, for example, the shelf life, weight of a fried food, temperature, humidity, size, arrangement of fried foods during deep-fry cooking, thickness, ratio of batter-coating, or a combination thereof may be considered.

Furthermore, the determination device may be configured to estimate a level of deterioration of edible oil.

The result of estimation may be output in the format of indicating the tendency of deterioration or in the format in which when to change the edible oil, such as whether it is time to change the edible oil, is estimated.

For example, the result of estimation is displayed on the monito in the format like “current level of deterioration is 00%”. That is, the monitor shows the current level expressed by a percentage where the percentage of the time in the future to change the oil is “100%”. On the other hand, when the level of deterioration shows the time to change has been reached, the monitor may display, for example, the result of determination by displaying a message like “please change frying oil”.

When the type of fried food to be deep-fried next and the number of pieces for each type are input or estimated, the monitor displays, for example, “0 more pieces to be deep-dried are left”, “you can deep-fry 0 pieces of ∘∘ or ●● pieces of for next time”, “add new oil now, and you can use this oil for ∘ more days”, and the like. That is, the monitor may display, based on the result of determination by the determination device, the details capable of being cooked until the time to change the oil has been reached, which are, for example, the type and number of fried foods.

Analyzing an image may enable calculation of the “number of bubbles”, “size of a bubble”, “ratio of the area where bubbles each having the predetermined size are formed relative to the total area”, “time from formation of the specific bubbles to disappearance thereof (speed of disappearance)”, or a combination thereof.

Furthermore, using these results of calculation, images, or a combination thereof may enable identification of the “acid value”, “color tone”, “rate of increase in viscosity”, “degree of flow of bubbles”, “visibility of the outline of an object to be cooked within an image”, type of frying oil, type of a fried food, quantity of fried foods, a combination thereof, or the like.

### OTHER EMBODIMENTS

In the examples described above, the determination device performs both the prior processing on the learning model and the execution processing using the learned model. However, the prior processing and the execution processing may not be performed by the same information processing device. Furthermore, each of the prior processing and the execution processing may not be executed consistently in one information processing device. In other words, each of the processing, storing data, and the like may be performed by an information system or the like including a plurality of information processing devices.

Note that the determination device or the like may further perform additional learning after the execution processing or before the execution processing.

Other embodiments in which the embodiments described above are combined each other may be adopted.

In an exemplary embodiment, the process of reducing over-learning (also referred to as “over-fitting”) such as dropout may be performed. In addition, the pre-processes such as dimension reduction and normalization may be performed.

The network architectures of the learning model and learned model may be CNN. For example, the network architecture may have a configuration such as RNN (Recurrent Neural Network) or LSTM (Long Short-Term Memory). In other words, the network architecture of AI may be other than deep-learning.

Furthermore, the learning model and the learned model may have a configuration including hyper parameters. That is, the learning model and the learned model may allow a user to set a part of the settings. Still further, AI may identify the amount of feature to be trained, or the user may set some or all of the amount of feature to be trained.

Still further, the learning model and the learned model may use other types of machine learning. For example, the learning model and the learned model may perform the pre-processing such as normalization by using an unsupervised model. Furthermore, learning may be reinforcement learning, or the like.

In learning, data expansion or the like may be performed. In other words, in order to increase the training data to be used in learning of the learning model, a pre-processing for expanding one piece of experiment data into a plurality of pieces of learning data may be performed. Thus, increasing the training data can advance the learning of the learning model more.

The present invention may be implemented by the determination and learning method exemplified above or a program for executing the processing equivalent to the processing described above (including firmware and one equivalent to the program, and hereinafter, simply referred to as “program”).

That is, the present invention may be realized by a program or the like described in a programming language or the like so that a predetermined result is obtained by executing a command to a computer. The program may be configured to execute a part of the processing by hardware such as an integrated circuit (IC) or a computing device such as a GPU.

The program causes a computer to execute the processing described above by making the computing device, control device, and storage device equipped in the computer cooperate. That is, the program is loaded onto the main storage device or the like and then issues a command to cause the computing device to execute the calculation, thereby causing the computer to operate.

Furthermore, the program may be provided via a computer-readable recording medium or a telecommunication line such as a network.

The present invention may be realized by a system including a plurality of devices. That is, the information processing system by a plurality of computers may execute the processing described above by a redundant, parallel, or distributed system, or a combination thereof. Accordingly, the present invention may be realized by a device having a configuration other than the hardware configuration described above and a system other than the one described above.

In the above, the present invention has been described with reference to the embodiments of the present invention. The present invention is not limited to the embodiments described above, and various modifications may be made therein. For example, each of the embodiments is described in detail herein for the purpose of clarity and a concise description, and the present invention is not necessarily limited to those including all the features described above.

Furthermore, some of the features according to a predetermined embodiment can be replaced with other features according to the separate embodiments, and other features can be added to the configuration of a predetermined embodiment. Still further, some of the features can include other features of the separate embodiments, be deleted, and/or replaced.

### REFERENCE SIGNS LIST

5: determination device
5F1: imaging section
5F2: first input section
5F3: first identification section
5F4: second identification section
5F5: second input section
5F6: output section
5F7: adjustment section
5F8: cooking environment input section
5F9: generation section
6: learning device
7: determination system
24: scale
41: monitor
42: video camera
51: adjustment unit
200: information system
300: network
A1: learning model
A2: learned model
D11: learning data
D111: amount of oil
D112: first information
D113: cooking temperature
D12: input data
D121: unlabeled amount of oil
D122: unlabeled first information
D13: estimation result
D22: table
IMG: image
L1: input layer
L2: intermediate layer
L3: output layer
W: waste oil
X: fried food
Y: frying oil
Z: disposal company

1. A determination device for determining a cooking environment of an edible oil, comprising:
an imaging section configured to acquire an image in which the edible oil is captured;
a first input section configured to input first information which is information on a fried food to be fed into the edible oil and cooked;
a first identification section configured to analyze the image and identify a state of the edible oil; and
a second identification section configured to identify the cooking environment in which the fried food is to be cooked, based on the first information and the state.

2. The determination device according to claim 1, further comprising a second input section configured to input second information indicating an amount of additional oil to be added to the edible oil, an amount of waste oil to be disposed from the edible oil, or a combination thereof.

3. The determination device according to claim 1, wherein
the cooking environment includes a temperature allowing the fried food to be cooked, an amount of decrease in temperature when the fried food is fed into the edible oil, a level of deterioration of the edible oil, or a combination thereof.

4. The determination device according to claim 3, wherein
the level of deterioration of the edible oil includes an acid value of the edible oil, a viscosity of the edible oil, a rate of increase in viscosity of the edible oil, a color tone of the edible oil, an anisidine value of the edible oil, an amount of polar compound of the edible oil, a carbonyl value of the edible oil, a smoke point of the edible oil, or an amount of volatile component of the edible oil.

5. The determination device according to claim 1, wherein
the state includes an amount of the edible oil, a difference from an optimum amount of the edible oil, a temperature of the edible oil, or a combination thereof.

6. The determination device according to claim 1, wherein
the second identification section is configured to identify the cooking environment using either of:
a table indicating a relation between input data, which includes a combination of the first information and the state, and the cooking environment; or
a learned model in which the relation between the input data and the cooking environment is learned by machine-learning.

7. The determination device according to claim 1, wherein
the first information is information indicating a type of the fried food, a volume of the fried food to be fed into the edible oil, or a combination thereof.

8. The determination device according to claim 1, wherein
the second identification section is configured to further identify a good taste of the fried food when the fried food is cooked in the cooking environment, and
the determination device further comprises an output section configured to output the cooking environment, an amount of additional oil to be added to the edible oil for making the good taste optimized, an amount of waste oil to be disposed from the edible oil, a first timing for adding the additional oil, a second timing for disposing the waste oil, or a combination thereof, based on the cooking environment or a result of identification of the good taste.

9. The determination device according to claim 8, further comprising an adjustment section configured to add the edible oil, dispose the edible, or both based on a result of output from the output section.

10. The determination device according to claim 1, wherein
the state incudes a first amount of oil indicating an amount of edible oil,
the first identification section is configured to:
identify an expansion rate of the edible oil; and
correct the first amount of oil based on the expansion coefficient to identify a second amount of oil, and
the second identification section is configured to identify the cooking environment based on the first information and the second amount of oil.

11. The determination device according to claim 1, wherein
the second identification section is configured to identify the cooking environment based on a correlation between the first information and the state, and the cooking environment.

12. A learning device for causing a learning model to learn so as to generate a learned model for determining a cooking environment of an edible oil, comprising:
an imaging section configured to acquire an image in which the edible oil is captured;
a first input section configured to input first information which is information on a fried food to be fed into the edible oil and cooked;
a first identification section configured to analyze the image and identify a state of the edible oil;
a cooking environment input section configured to input the cooking environment in which the fried food is to be cooked; and
a generation section configured to input the first information, the state, and the cooking environment and cause the learning model to learn so as to generate the learned model.

13. A determination system comprising:
the determination device according to claim 1; and
the learning device according to claim 12.

14. A determination method of determining a cooking environment of an edible oil, comprising:
an imaging step of acquiring an image in which the edible oil is captured;
a first input step of inputting first information which is information on a fried food to be fed into the edible oil and cooked;
a first identification step of identifying a state of the edible oil by analyzing the image; and
a second identification step of identifying the cooking environment in which the fried food is to be cooked, based on the first information and the state.

15. A program for making a computer execute the determination method according to claim 14.

16. A learning method of generating a learned model for determining a cooking environment of an edible oil by causing a learning model to learn, comprising:
an imaging step of acquiring an image in which the edible oil is captured;
a first input step of inputting first information which is information on a fried food to be fed into the edible oil and cooked;
a first identification step of identifying a state of the edible oil by analyzing the image;
a cooking environment input step of inputting the cooking environment in which the fried food is to be cooked; and
a generation step of generating the learned model by inputting the first information, the state, and the cooking environment and causing the learning model to learn.

17. A computer-readable media for making a computer execute the learning method according to claim 16.
